(19) United States
(12) Patent Application Publication
(10) Pub. No.: US 2010/0056867 A1
(43) Pub. Date: Mar. 4, 2010

(54) Endoscope Valve

(75) Inventors: Denis Labombard, Georgetown, MA (US); Mark Landman, Sharon, MA (US); Thomas S. Soulos, New York, NY (US)

Correspondence Address: MARTIN D. MOYNIHAN d/b/a PRTSI, INC., P.O. BOX 16446, ARLINGTON, VA, 22215, US

(73) Assignee: Vision - Sciences Inc., Orangeburg, NY (US)

(21) Appl. No.: 11/792,482

(22) PCT Filed: Dec. 8, 2005
(86) PCT No.: PCT/US05/44650

§ 371 Date: Nov. 9, 2009

Related U.S. Application Data
(60) Provisional application No. 60/634,117, filed on Dec. 8, 2004.

(51) Int. Cl. A61B 1/12; A61B 1/307

(52) U.S. Cl. 600/121; 600/159; 600/135

(57) ABSTRACT

An endoscope including a handle and an insertion tube, adapted for insertion into a patient, connected to the handle. In addition, the endoscope includes at least one valve, mounted on the handle, for controlling flow through one or more channels, which valve has at least two stable states. In the stable states the valve does not apply a force to leave the state.

### RELATED APPLICATIONS

This application claims the benefit under 119(e) of U.S. provisional patent application 60/634,117, filed Dec. 8, 2004, the disclosure of which is incorporated herein by reference.

### FIELD OF THE INVENTION

The present invention relates to apparatus for minimally invasive procedures and particularly to valves of minimally invasive probes.

### BACKGROUND OF THE INVENTION

Minimally invasive probes, such as endoscopes, are used for many surgical procedures, in order to reduce the trauma in full scale surgical operations. Endoscopes may include in their small diameter a plurality of separate apparatus, including, for example, two or more of viewing, suction and irrigation apparatus. In order to reduce costs and prevent mistakes due to misunderstandings between members of a medical staff, it is advantageous that a single physician controls operation of all the apparatus of the endoscope. Such single operator control requires proper human engineering of the controls of the endoscope.

U.S. Pat. No. 4,852,551 to Opie et al., the disclosure of which is incorporated herein by reference, describes a contamination-free endoscope pinch valve for use with a disposable endoscope sheath. The valve is located on a handle of the endoscope and is actuated by pushing a button. The valve includes a spring that biases the valve in a specific state.

U.S. Pat. No. 6,666,818 to Dhindsa, the disclosure of which is incorporated herein by reference, describes an endoscope valve assembly that is releasably attached to the endoscope handle. The valve is biased in an upper position by a spring. A latch is used to keep the valve in the open position until it is released by the user.

### SUMMARY OF THE INVENTION

An aspect of some embodiments of the present invention relates to an endoscope tube valve mounted on the handle of an endoscope, which valve has at least two stable states (i.e., states in which the valve does not apply force urging the valve to leave the state, e.g., by a biasing spring). The at least two states optionally include states in which the level of blocking of the tube is different. In some embodiments of the invention, the at least two states include at least one state in which flow through the tube is at least partially blocked (for example, substantially entirely blocked) and at least one state in which flow through the tube is not blocked by the valve. Alternatively or additionally, the at least two states include a first stable state in which the open cross section of the tube is at least twice or even at least four times the open cross section in a second stable state.

In some embodiments of the invention, the valve does not have any state in which the valve itself applies a force which urges the valve into a different state. The use of a valve which does not have a preferred state allows simpler control of the valve by a physician.

In some embodiments of the invention, gravitational forces on the valve are not strong enough to change the functional state of the valve (i.e., the level of blocking of the tube), in at least one or even two of the functional states of the valve.

In some embodiments of the invention, in one or more of the stable states, the valve applies a retaining force when an attempt is made to remove it from the stable state. Thus, leaving the stable state requires application of at least a predetermined force. Optionally, the retaining force feature is activated by the movement of the valve into the stable state, without requiring an additional latching act by a human operator of the valve.

In some embodiments of the invention, the valve comprises a pinch valve. Optionally, the valve is an integral part of the endoscope, while the tube is part of a disposable sheath attached to the endoscope.

There is therefore provided in accordance with an exemplary embodiment of the invention, an endoscope, comprising a handle, an insertion tube, adapted for insertion into a patient, connected to the handle, at least one valve, mounted on the handle, for controlling flow through one or more channels, which valve has at least two stable states, wherein in the stable states the valve does not apply a force to leave the state.

Optionally, the at least one valve controls the flow through a channel, which belongs to a sheath covering the insertion tube. Optionally, the at least one valve includes a niche adapted to receive a channel to be controlled by the valve. Optionally, in at least one of the stable states it is required to apply at least a predetermined force to leave the state.

Optionally, the valve comprises a pinch valve. Optionally, in at least one of the stable states, the valve applies a force which counters exiting the state. Optionally, the movement of the valve into the stable state such that it does not exit the state does not require additional acts beyond bringing the valve to the position of the stable state. Optionally, the valve comprises a base and a handle which rotates around a pivot attached to the base. Optionally, the handle comprises a pinching roller adapted to block the flow through the one or more channels.

Optionally, the handle includes a plurality of valves mounted thereon. Optionally, substantially all the possible states of the valve are stable. Optionally, the valve has at least five different stable states.

There is further provided in accordance with an exemplary embodiment of the invention, an endoscope, comprising a handle, an insertion tube, adapted for insertion into a patient, connected to the handle, at least one valve, mounted on the handle, for controlling flow through at least two channels, which valve includes a member moveable between at least two states, the member limiting the flow through a first channel in a first one of the states and limiting the flow through a second channel in a second one of the states.

Optionally, the member has a third state in which it does not block the flow in any of the channels. Optionally, the member does not have a state in which it blocks two or more of the channels at once. Optionally, the endoscope comprises a cystoscope. Optionally, the member comprises a roller. Optionally, the member moves between states by rotation. Optionally, the member moves between states by linear movement.

### BRIEF DESCRIPTION OF THE DRAWINGS

Exemplary non-limiting embodiments of the invention will be described with reference to the following description of the embodiments, in conjunction with the figures. Identical structures, elements or parts which appear in more than one figure are preferably labeled with the same or similar number in all the figures in which they appear, and in which:

FIG. 1A is a schematic view of an endoscope, in accordance with an exemplary embodiment of the invention;

FIG. 1B is a schematic sectional view of an endoscope with a sheath, in accordance with an exemplary embodiment of the invention;

FIG. 2A is a side view of a toggle valve, in accordance with an exemplary embodiment of the invention;

FIG. 2B is a perspective view of the toggle valve of FIG. 2A, in accordance with an exemplary embodiment of the invention;

FIG. 3 is a cross sectional view of the valve of FIG. 2A, in accordance with an exemplary embodiment of the invention; and

FIG. 4 is a schematic cross sectional view of a switch, in accordance with another exemplary embodiment of the invention.

### DETAILED DESCRIPTION OF EXEMPLARY EMBODIMENTS

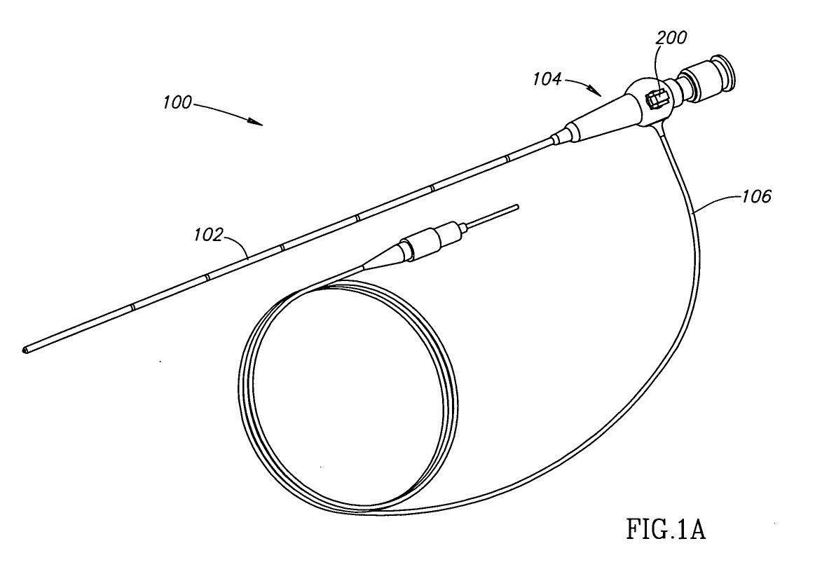

FIG. 1A is a schematic view of an endoscope 100, in accordance with an exemplary embodiment of the invention. Endoscope 100 includes an insertion tube 102, for insertion into a patient and a handle 104 for control of the insertion tube and/or for holding the endoscope by a physician. Insertion tube 102 may be adapted for insertion into any body cavity with which endoscopes are used, including, for example, the esophagus, colon and/or urethra. In an exemplary embodiment of the invention, endoscope 100 includes a cystoscope. An umbilical 106 optionally connects handle 104 to a light source (not shown).

A valve 200 mounted on handle 104 is used to control the flow through a channel of a sheath covering endoscope 100, as described below. Alternatively to mounting only a single valve 200 on handle 104, a plurality of identical or different valves may be mounted on handle 104, for example for water, suction and/or air channels. The use of non-identical valves, for example having different form factors, reduces the chances of a physician from getting mixed up between the valves.

FIG. 1B is a schematic sectional view of endoscope 100 with a sheath 125 including a channel tube 130 therein, in accordance with an exemplary embodiment of the invention. Sheath 125 optionally covers insertion tube 102 and at least a portion of handle 104. Channel tube 130 optionally extends proximally beyond sheath 125, for connection to a control station 160, which serves, for example, as a source of fluid (e.g., liquid and/or air) under positive or negative pressure. Channel tube 130 is passed through valve 200 along its path between control station 160 and the distal end of insertion tube 102, so that a physician holding the endoscope by handle 104 can easily control the flow through tube 130, without aid from another person. Valve 200 is optionally a pinch valve which does not come in contact with fluids passing through channel tube 130 and therefore does not require frequent disinfection.

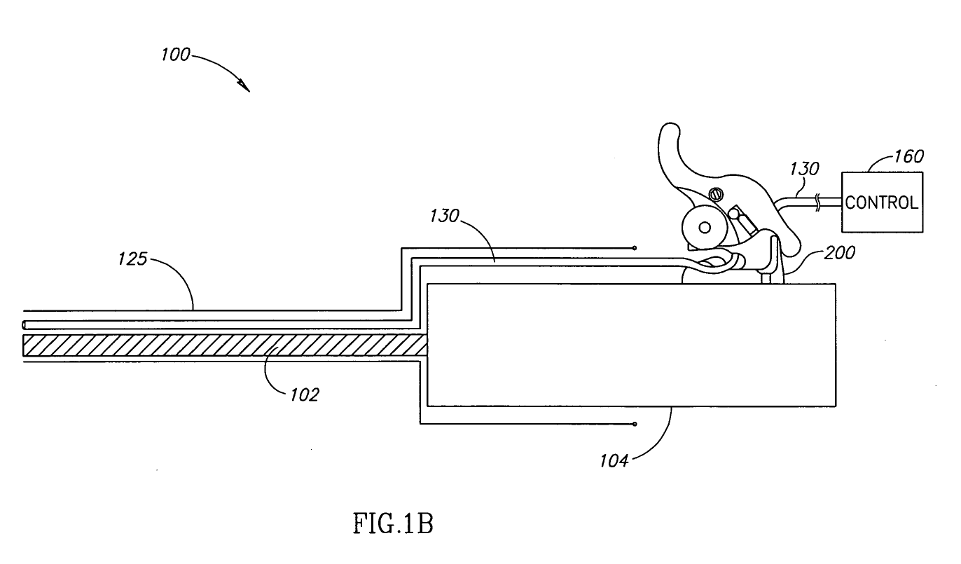

FIGS. 2A and 2B show, in side and perspective views respectively, a toggle valve 200, in accordance with an exemplary embodiment of the invention. Valve 200 comprises a base 202 and a toggle lever 204. Toggle lever 204 moves relative to base 202, for example, on a pivot 206. Base 202 optionally includes a lower portion 210, which is coupled to handle 104 (FIG. 1A) of the endoscope, and two parallel walls 212, on opposite sides of toggle lever 204. Walls 212 define a slot 214 through which one or more channel tubes 130 (FIG. 1B) to be controlled by valve 200, are inserted into a tube holding niche 216. Slot 214 optionally has a narrow bottle neck 218, which prevents inadvertent escape of tubes 130 from niche 216.

Lever 204 optionally includes a pinching element, such as a pinching roller 220 which presses on tubes 130 within niche 216, when lever 204 is rotated in a first direction. In some embodiments of the invention, lever 204 includes two extensions 222 and 224 which are pressed down by the physician alternately in order to change the state of the valve (e.g., between closed and open states). Lever 204 is optionally of a size which is convenient for use by physicians, for example, by pressing with the thumb or other finger of the physician.

In use, one or more channels to be controlled by valve 200 are passed through slot 214 into niche 216. When the physician wants to block the passage through the channel, extension 222 is pushed down, causing lever 204 to rotate around pivot 206 and thus push pinching roller 220 against the channel. In order to open the channel, the physician presses extension 224 downwards, thus causing pinching roller 220 to move away from the channel in niche 216 and allow flow through the channel.

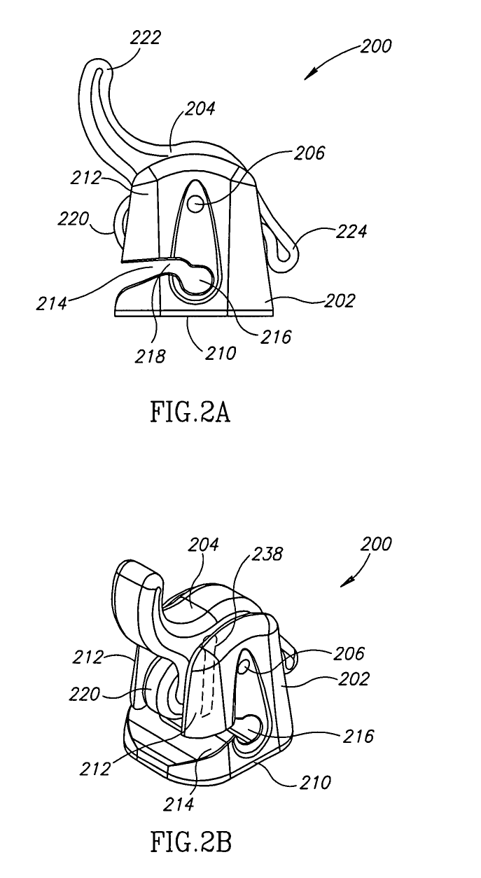

FIG. 3 is a sectional view of valve 200, in accordance with an exemplary embodiment of the invention. In some embodiments of the invention, a spring-loaded ball detent is used to keep valve 200 in one or more stable states. A male member 240 of the ball detent optionally mates with one or more grooves 238 (FIG. 2B) on the inner side of one or more of walls 212, when lever 204 is in one or more states (e.g., predetermined angles relative to base 202). The ball detent may be used to hold slot 214 open (e.g., for ease of installation of tube 130 into niche 216), closed and/or in any intermediate stable state. In place of groove 238, other shapes of holding cavities may be used.

Alternatively or additionally to using the ball detent, the movement of lever 204 around pivot 206 has a constant friction, which requires a physician to apply a force of at least a predetermined level in order to move lever 204. The friction force is optionally larger than any force on the valve due to gravity, resilience of tube 130 and/or pressure in tube 130. In this embodiment, substantially all states of the valve are stable.

Further alternatively or additionally, lever 204 is rotatable around pivot 206 until a maximal rotation point of a closed state, in which pinching roller 220 is pushed beyond a maximal compression point of channel tube 130. At this state, the release of lever 204 from the closed state requires applying force, to further compress channel tube 130 before it is released.

Further alternatively or additionally, any other retention mechanism holds valve 200 in one or more of the stable states. In some embodiments of the invention, different types of retention mechanisms are used in holding valve 200 in at least two different stable states. In other embodiments, the same type of retention mechanism (e.g., friction) is used to hold valve 200 in a plurality of different stable states.

A set screw 250 optionally holds pivot 206 in place and hence keeps lever 204 and base 202 in an assembled state.

Pinching roller 220 is optionally mounted rotateably on a shaft 236 of lever 204, so that pinching roller 220 does not damage channel tube 130 when it is pressed against the tube. Alternatively, for simplicity, pinching roller 220 is an integral part of lever 204 and is not a roller, but rather an appendage of lever 204.

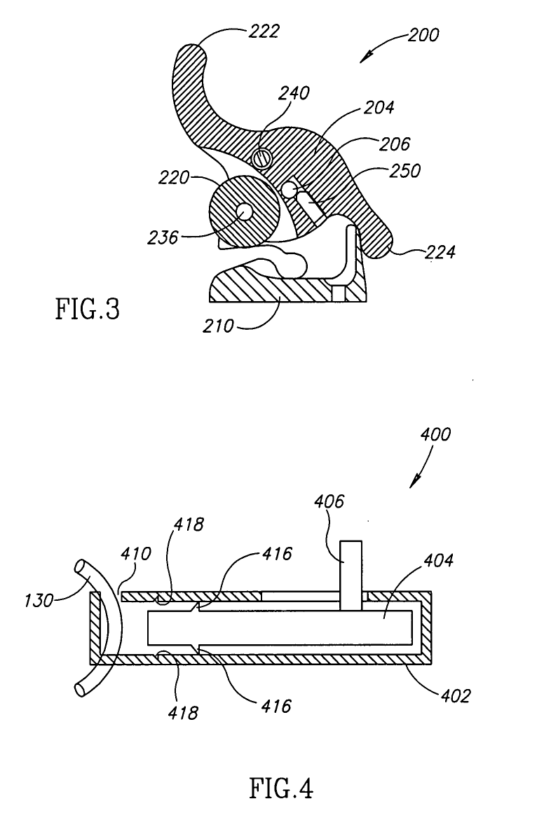

FIG. 4 is a schematic cross sectional view of a switch 400, in accordance with another exemplary embodiment of the invention. Switch 400 includes a base 402, which serves as a housing for a moving bar 404. A slit 410 in base 402 allows insertion of a channel tube 130 into the movement path of bar 404. Bar 404 may be moved between a state in which it does not apply force on tube 130 and a state in which it substantially prevents flow through the tube.

Moving bar 404 optionally includes a handle 406 which is used by a physician to move bar 404.

In some embodiments of the invention, moving bar 404 includes one or more protrusions 416, which fit into respective notches 418 in base 402. The fitting of protrusions 416 into their respective notches 418, prevents inadvertent movement of bar 404 out of its current state. That is, in order to take switch 400 out of its closed stable state, at least a minimal force is required. In some embodiments of the invention, base 402 includes only a single set of one or more notches corresponding to protrusions 416. For example, the set of notches may be used for keeping the channel tube 130 in a state which prevents flow, against the resistance of the tube 130. Alternatively, base 402 includes a plurality of sets of notches 418, for example for a closed state, in which no flow passes through tube 130, a half closed state and an open state. In some embodiments of the invention, a same threshold force is defined for moving switch 400 out of the stable state. Alternatively, different states have different threshold forces required for moving them out of their stable state.

The protrusions 416 are optionally elastically and/or spring loaded on bar 404, so as to allow easy entrance into and release from notches 418. Alternatively or additionally, the movement of bar 404 is damped, for example by a friction or hydraulic force, to prevent undesired inadvertent movement.

In some embodiments of the invention, valve 400 is mounted on handle 104 (FIG. 1A) of the endoscope in an orientation such that bar 404 is moved in parallel to the handle. Alternatively, bar 404 is moved toward and away from the handle or circumferentially around the handle. Further alternatively, any other orientation of valve 400 relative to handle 104 may be used.

Valves 200 and 400 may be used for a single channel tube 130. Alternatively, valves 200 and/or 400 may be used for a plurality of channel tubes which need to be opened and closed together. Further alternatively, the valves may be used with a pair of tubes, a first tube of which must be closed when the second tube is closed. The channel tubes are positioned such that in a half closed state, the first tube is closed while the second tube remains open. In a fully closed state, both of the tubes are closed.

In other embodiments of the invention, a valve is designed to receive two tubes in two different niches. A lever or bar may be moved to press on one or the other of the tubes but not on both tubes together. In some embodiments of the invention, at any time, one of the tubes is closed for flow. Alternatively, the valve has three states, a state in which both tubes are open and states in which one of the tubes is closed and the other is open.

In FIGS. 2A and 2B, roller 220 presses against tube 130 in an orientation in which tube 130 is perpendicular to the roller. In other embodiments of the invention, roller 220 presses against tube 130 in parallel to the axis of the tubes. In these embodiments, by using a wide roller, a plurality of tubes 130 laying next to each other can be controlled together.

It will be appreciated that the above-described apparatus and methods may be varied in many ways. It should also be appreciated that the above described description of methods and apparatus are to be interpreted as including apparatus for carrying out the methods, and methods of using the apparatus.

The present invention has been described using non-limiting detailed descriptions of embodiments thereof that are provided by way of example and are not intended to limit the scope of the invention. It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art. Furthermore, the terms “comprise,” “include,” “have” and their conjugates, shall mean, when used in the claims, “including but not necessarily limited to.”

It is noted that some of the above described embodiments may describe the best mode contemplated by the inventors and therefore may include structure, acts or details of structures and acts that may not be essential to the invention and which are described as examples. Structure and acts described herein are replaceable by equivalents which perform the same function, even if the structure or acts are different, as known in the art. Therefore, the scope of the invention is limited only by the elements and limitations as used in the claims.

1. An endoscope, comprising:
a handle;
an insertion tube, adapted for insertion into a patient, connected to the handle; and
at least one valve, mounted on the handle, for controlling flow through one or more channels, which valve has at least two stable states, wherein in the stable states the valve does not apply a force to leave the state.

2. An endoscope according to claim 1, wherein the at least one valve controls the flow through a channel, which belongs to a sheath covering the insertion tube.

3. An endoscope according to claim 1, wherein the at least one valve includes a niche adapted to receive a channel to be controlled by the valve.

4. An endoscope according to claim 1, wherein in at least one of the stable states it is required to apply at least a predetermined force to leave the state.

5. An endoscope according to claim 1, wherein the valve comprises a pinch valve.

6. An endoscope according to claim 1, wherein in at least one of the stable states, the valve applies a force which counters exiting the state.

7. An endoscope according to claim 1, wherein the movement of the valve into the stable state such that it does not exit the state does not require additional acts beyond bringing the valve to the position of the stable state.

8. An endoscope according to claim 1, wherein the valve comprises a base and a handle which rotates around a pivot attached to the base.

9. An endoscope according to claim 1, wherein the handle comprises a pinching roller adapted to block the flow through the one or more channels.

10. An endoscope according to claim 1, wherein the handle includes a plurality of valves mounted thereon.

11. An endoscope according to claim 1, wherein substantially all the possible states of the valve are stable.

12. An endoscope according to claim 1, wherein the valve has at least five different stable states.

13. An endoscope according to claim 1, wherein one of the at least one valve is adapted to control a plurality of channels.

14. An endoscope, comprising:
a handle;
an insertion tube, adapted for insertion into a patient, connected to the handle; and
at least one valve, mounted on the handle, for controlling flow through at least two channels, which valve includes a member moveable between at least two states, the member limiting the flow through a first channel in a first one of the states and limiting the flow through a second channel in a second one of the states.

15. An endoscope according to claim 14, wherein the member has a third state in which it does not block the flow in any of the channels.

16. An endoscope according to claim 14, wherein the member does not have a state in which it blocks two or more of the channels at once.

17. An endoscope according to claim 14, wherein the endoscope comprises a cystoscope.

18. An endoscope according to claim 14, wherein the member comprises a roller.

19. An endoscope according to claim 14, wherein the member moves between states by rotation.

20. An endoscope according to claim 14, wherein the member moves between states by linear movement.
